(19) United States
(12) Patent Application Publication
(10) Pub. No.: US 2026/0315042 A1
(43) Pub. Date: Oct. 15, 2026

(54) Toilet Stool Sampler

(71) Applicants: John Robert Kasha, Jr., North Potomac, MD (US); John Robert Kasha, III, North Potomac, MD (US)

(72) Inventors: John Robert Kasha, Jr., North Potomac, MD (US); John Robert Kasha, III, North Potomac, MD (US)

(21) Appl. No.: 19/466,313

(22) Filed: Jan. 31, 2026

Related U.S. Application Data
(60) Provisional application No. 63/752,723, filed on Feb. 1, 2025.

(51) Int. Cl. A61B 10/00

(52) U.S. Cl. CPC A61B 10/0038; A61B 2010/0003

(57) ABSTRACT

A stool sampling collector includes a platform and a movable cover and is placed in a toilet. An actuator is controlled to move the cover to a second end of the platform, providing an area of stool collection on the platform between the cover and one or more pipes extending from a first end of the platform. The actuator is controlled to move the cover to the first end to move the stool collected on the platform toward and into openings of the pipes until at least one of the surfaces of the cover is in contact with an opening of the pipes. A pump is controlled to provide suction to a collection vessel that, in turn, provides suction to one or more pipes that move stool from at least one pipe of the pipes to the collection vessel.

### RELATED APPLICATIONS

This application claims the benefit of U.S. Provisional Patent Application Ser. No. 63/752,723, filed on Feb. 1, 2025, the content of which is incorporated by reference herein in its entirety.

### FIELD OF THE INVENTION

The teachings herein relate to systems and methods for automatic stool sampling in a toilet. More particularly, the teachings herein relate to controlling a stool sample collector placed in a toilet to collect portions of stool and one or more pumps to move the collected portions of stool to a collection vessel.

### BACKGROUND

Smartphones have made it possible to monitor and control all kinds of nearby devices. Most recently, smartphones are being used to track a growing number of home-based health monitoring devices and tests. In an article, So A, the health monitoring boom only gets weirder from here, Wired, 2025 Jan. 7, (hereinafter the “So Article”) describes this growth in home-based and smartphone-connected health monitoring devices and tests as a boom.

The So Article reviews several home-based health monitoring devices and tests. These include a glucose monitor, an atrial health monitor, hormone monitoring saliva test strips, and women's health test strips. The article describes that many of these monitoring devices and tests are not invasive, but many are moving in that direction. The glucose monitor, for example, includes small filaments or needles that are inserted under the skin.

A recent paper suggests one important type of noninvasive home monitoring that remains largely unexplored. In a paper, Ge T J et al., passive monitoring by smart toilets for precision health, Sci Transl Med. 2023 Feb, (hereinafter the “Ge Paper”) describe the many benefits that could be derived from monitoring excretory material for what they call “precision health.”

The Ge Paper proposes that the bathroom, and more specifically the toilet, is well-suited for noninvasive health monitoring devices. The Ge Paper outlines a wealth of useful health information that can be obtained by monitoring and sampling urine and stool.

The Ge Paper suggests, however, that two factors are preventing urine and stool analysis from being a “gateway to the ‘digitalization’ of health care in the home.” First, they claim there is a “reluctance to openly discuss excreta.” Second, they claim that patients are generally “reluctant to handle their own excrement.” As evidence, they report that, despite manual home stool tests being shown to reduce colorectal cancer mortality, patient participation rates for stool testing remain at or below 30%. Our reluctance to handle stool is evolutionary. It was needed to prevent the spread of bacteria-borne disease.

The Ge Paper surmises that the use of smart toilets is the best way to automate urine and stool analyses and overcome the stigma associated with excretory data. By “smart toilet” they mean integrating computer-controlled and monitored sensors into toilet seats or entire toilets. This is similar to what is currently being done to enhance the “toileting experience” by adding bidets, heated seats, and other devices to toilet seats or entire toilets.

One exemplary smart toilet for automated urine and stool analyses is provided in U.S. Pat. No. 11,604,177 (hereinafter the “'177 Patent”). A stool analysis module of the '177 Patent includes one or more cameras placed in the toilet bowl for imaging stool to “identify colorimetric, fecal occult blood, and/or other changes for screening cancer and/or other conditions.”

The Ge Paper also references a system and method that can provide sample collection by modifying the plumbing connected to a toilet. This reference is Grego S et al., a hands-free stool sampling system for monitoring intestinal health and disease, Sci Rep. 2022 Jun. 27 (hereinafter the “Grego Paper”). The Grego Paper also suggests that important health information can be obtained by monitoring and sampling stool and highlights the fact that only a small amount of material is needed to obtain this information. To get this material, the Grego Paper describes a system and method for collecting stool samples after a toilet is flushed and the stool enters a drain pipe. The system and method of the Grego Paper are also described in U.S. Patent Publication 2023/0102589 (hereinafter the “'589 Publication”).

Through a series of valves added to the drain pipe of a toilet, the system of the '589 Publication immobilizes stool in the pipe. The system then liquefies the immobilized stool using a jet of liquid sprayed at the stool. A valve is then opened to collect the liquified stool in a sample extraction vessel.

In summary, the So Article shows the current and growing interest in home-based health monitoring devices. The Ge Paper and the Grego Paper stress the importance of extending home-based health monitoring and sampling to the bathroom of the home.

One problem, however, with the system of the '177 Patent is outlined in the Ge Paper. The paper describes that it is not clear that patients will adopt the use of cameras and other types of sensors in their toilets due to privacy concerns.

A problem with both the system of the '177 Patent and the system of the '589 Publication is that they are both complex and expensive for a home-based device. The system of the '177 Patent relies on cameras and sophisticated image processing to produce actionable health information. The system of the '589 Publication requires significant modification to the plumbing of a house. It will also likely require modifications to the walls of the house to access and maintain the pipes and valves added.

As a result, there is an unmet need for systems and methods that provide for stool collection in an existing toilet with little modification to the toilet and using inexpensive and readily available parts unlikely to cause privacy concerns.

### BRIEF DESCRIPTION OF THE DRAWINGS

The skilled artisan will understand that the drawings, described below, are for illustration purposes only. The drawings are not intended to limit the scope of the present teachings in any way.

FIG. 1 is a block diagram that illustrates a computer system, upon which embodiments of the present teachings may be implemented.

FIG. 2 is an exemplary diagram showing an automatic stool sampling system in which the stool sample collector is in the open position, in accordance with various embodiments.

FIG. 3 is an exemplary perspective view of the opened stool sample collector from the back and above, in accordance with various embodiments.

FIG. 4 is an exemplary perspective view of the closed stool sample collector from the back and above, in accordance with various embodiments.

FIG. 5 is an exemplary diagram showing a perspective view of the opened stool sample collector from the left and back, in accordance with various embodiments.

FIG. 6 is an exemplary diagram showing a perspective view of the closed stool sample collector from the left and back, in accordance with various embodiments.

FIG. 7 is an exemplary diagram showing that the system of FIG. 2 can include an additional pump for purging water from hoses, in accordance with various embodiments.

FIG. 8 is an exemplary diagram showing the system of FIG. 2 after a sample has been collected, in accordance with various embodiments.

FIG. 9 is an exemplary perspective view of the closed stool sample collector from the back and below, in accordance with various embodiments.

FIG. 10 is an exemplary perspective view of the closed stool sample collector from the back and below, in accordance with various embodiments.

FIG. 11 is an exemplary perspective view of the closed stool sample collector from the top, in accordance with various embodiments.

FIG. 12 is an exemplary perspective view of the open stool sample collector from the top, in accordance with various embodiments.

FIG. 13 is an exemplary perspective view of the closed stool sample collector from the bottom, in accordance with various embodiments.

FIG. 14 is an exemplary perspective view of the open stool sample collector from the bottom, in accordance with various embodiments.

FIG. 15 is an exemplary perspective view of the closed stool sample collector from the right side, in accordance with various embodiments.

FIG. 16 is an exemplary perspective view of the open stool sample collector from the right side, in accordance with various embodiments.

FIG. 17 is an exemplary perspective view of the closed stool sample collector from the left side, in accordance with various embodiments.

FIG. 18 is an exemplary perspective view of the open stool sample collector from the left side, in accordance with various embodiments.

FIG. 19 is an exemplary flowchart showing a method 1900 for automatic stool sampling in a toilet, in accordance with various embodiments.

Before one or more embodiments of the present teachings are described in detail, one skilled in the art will appreciate that the present teachings are not limited in their application to the details of construction, the arrangements of components, and the arrangement of steps set forth in the following detailed description or illustrated in the drawings. Also, it is to be understood that the phraseology and terminology used herein are for the purpose of description and should not be regarded as limiting.

### DESCRIPTION OF VARIOUS EMBODIMENTS

### Computer-Implemented System

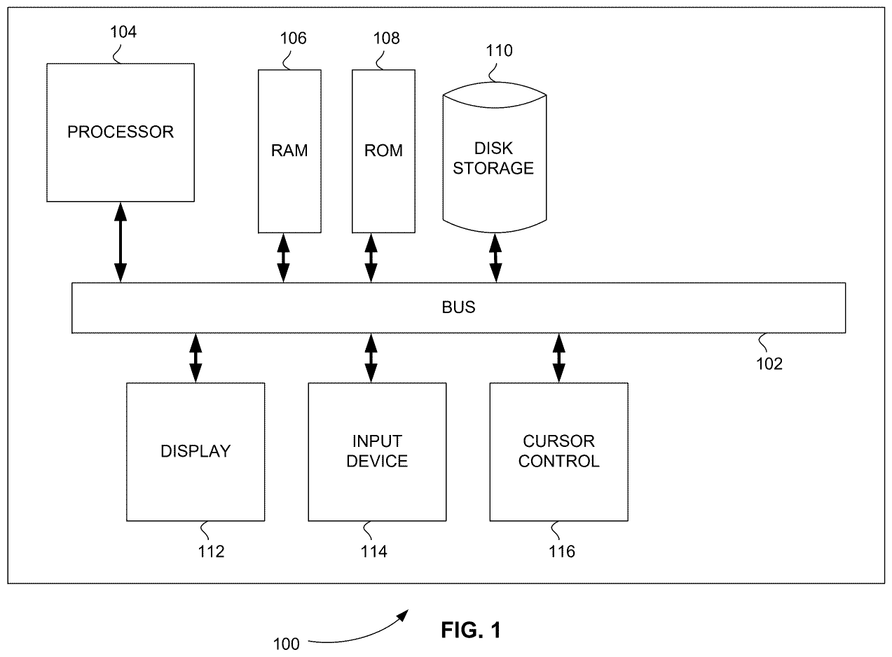

FIG. 1 is a block diagram that illustrates a computer system 100, upon which embodiments of the present teachings may be implemented. Computer system 100 includes a bus 102 or other communication mechanism for communicating information, and a processor 104 coupled with bus 102 for processing information. Computer system 100 also includes a memory 106, which can be a random-access memory (RAM) or other dynamic storage device, coupled to bus 102 for storing instructions to be executed by processor 104. Memory 106 also may be used for storing temporary variables or other intermediate information during execution of instructions to be executed by processor 104. Computer system 100 further includes a read only memory (ROM) 108 or other static storage device coupled to bus 102 for storing static information and instructions for processor 104. A storage device 110, such as a magnetic disk or optical disk, is provided and coupled to bus 102 for storing information and instructions.

Computer system 100 may be coupled via bus 102 to a display 112, such as a cathode ray tube (CRT) or liquid crystal display (LCD), for displaying information to a computer user. An input device 114, including alphanumeric and other keys, is coupled to bus 102 for communicating information and command selections to processor 104. Another type of user input device is cursor control 116, such as a mouse, a trackball or cursor direction keys for communicating direction information and command selections to processor 104 and for controlling cursor movement on display 112.

A computer system 100 can perform the present teachings. Consistent with certain implementations of the present teachings, results are provided by computer system 100 in response to processor 104 executing one or more sequences of one or more instructions contained in memory 106. Such instructions may be read into memory 106 from another computer-readable medium, such as storage device 110. Execution of the sequences of instructions contained in memory 106 causes processor 104 to perform the process described herein.

Alternatively, hard-wired circuitry may be used in place of or in combination with software instructions to implement the present teachings. For example, the present teachings may also be implemented with programmable artificial intelligence (AI) chips with only the encoder neural network programmed-to allow for performance and decreased cost. Thus, implementations of the present teachings are not limited to any specific combination of hardware circuitry and software.

The term “computer-readable medium” or “computer program product” as used herein refers to any media that participates in providing instructions to processor 104 for execution. The terms “computer-readable medium” and “computer program product” are used interchangeably throughout this written description. Such a medium may take many forms, including but not limited to, non-volatile media and volatile media. Non-volatile media includes, for example, optical or magnetic disks, such as storage device 110. Volatile media includes dynamic memory, such as memory 106.

Common forms of computer-readable media include, for example, a floppy disk, a flexible disk, hard disk, magnetic tape, or any other magnetic medium, a CD-ROM, digital video disc (DVD), a Blu-ray Disc, any other optical medium, a thumb drive, a memory card, a RAM, PROM, and EPROM, a FLASH-EPROM, any other memory chip or cartridge, or any other tangible medium from which a computer can read.

Various forms of computer readable media may be involved in carrying one or more sequences of one or more instructions to processor 104 for execution. For example, the instructions may initially be carried on the magnetic disk of a remote computer. The remote computer can load the instructions into its dynamic memory and send the instructions over a telephone line using a modem. A modem local to computer system 100 can receive the data on the telephone line and use an infra-red transmitter to convert the data to an infra-red signal. An infra-red detector coupled to bus 102 can receive the data carried in the infra-red signal and place the data on bus 102. Bus 102 carries the data to memory 106, from which processor 104 retrieves and executes the instructions. The instructions received by memory 106 may optionally be stored on storage device 110 either before or after execution by processor 104.

In accordance with various embodiments, instructions configured to be executed by a processor to perform a method are stored on a computer-readable medium. The computer-readable medium can be a device that stores digital information. The computer-readable medium is accessed by a processor suitable for executing instructions configured to be executed.

The following descriptions of various implementations of the present teachings have been presented for purposes of illustration and description. It is not exhaustive and does not limit the present teachings to the precise form disclosed. Modifications and variations are possible in light of the above teachings or may be acquired from practicing of the present teachings. Additionally, the described implementation includes software but the present teachings may be implemented as a combination of hardware and software or in hardware alone. The present teachings may be implemented with both object-oriented and non-object-oriented programming systems.

### System for Stool Sampling

As described above, there is a current and growing interest in home-based health monitoring devices. The Ge Paper and the Grego Paper stress the importance of extending home-based health monitoring and sampling to the home bathroom.

One problem with the system of the '177 Patent is outlined in the Ge Paper. They describe that it is not clear whether patients will adopt the use of cameras in their toilets due to privacy concerns.

A problem with both the system of the '177 Patent and the system of the '589 Publication is that they are both overly complex and expensive. The system of the '177 Patent relies on cameras and sophisticated image processing to produce actionable health information. The system of the '589 Publication requires significant modification to the plumbing of a house.

As a result, there is an unmet need for systems and methods that provide for stool collection in an existing toilet with little modification to the toilet and using inexpensive and readily available parts unlikely to cause privacy concerns.

In various embodiments, a system for automatic stool sampling is added to an existing toilet. The system includes a stool sample collector. One or more pumps collect a stool sample from the stool sample collector automatically and place the sample in a collection vessel or cup. The stool sample collector and the one or more pumps are controlled by one or more processors. As a result, the stool sample can be collected without the user having to handle their stool or even see the stool being collected.

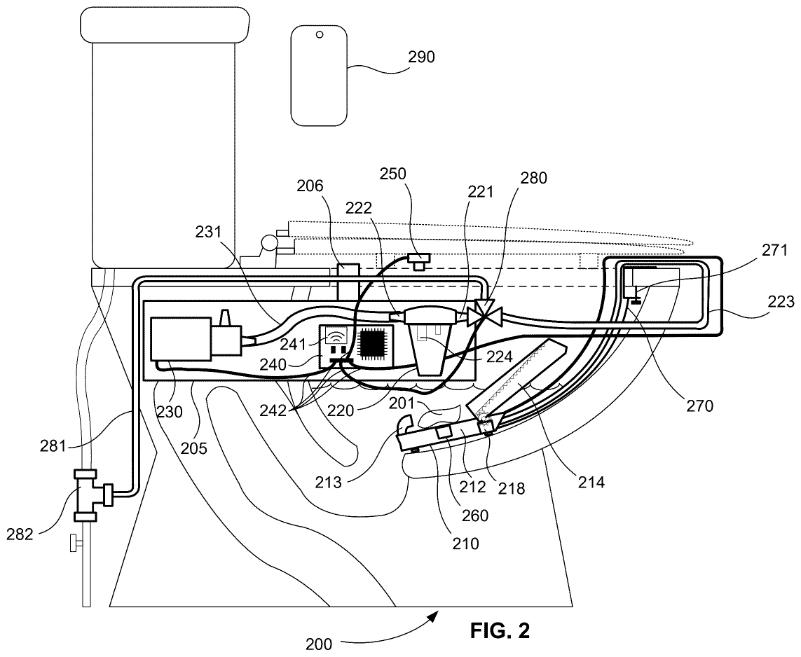

FIG. 2 is an exemplary diagram 200 showing an automatic stool sampling system in which the stool sample collector is in the open position, in accordance with various embodiments. The system of FIG. 2 includes stool sample collector 210, collection vessel 220, one or more pumps 230, and one or more processors 240.

Stool sample collector 210 includes a platform 212, a cover 214, and one or more actuators 218. Platform 212 includes one or more pipes 213 extending out of platform 212 at a first end of platform 212. One or more pipes 213 are shaped back towards platform 212 so that an axis of an opening of each pipe of one or more pipes 213 is above platform 212.

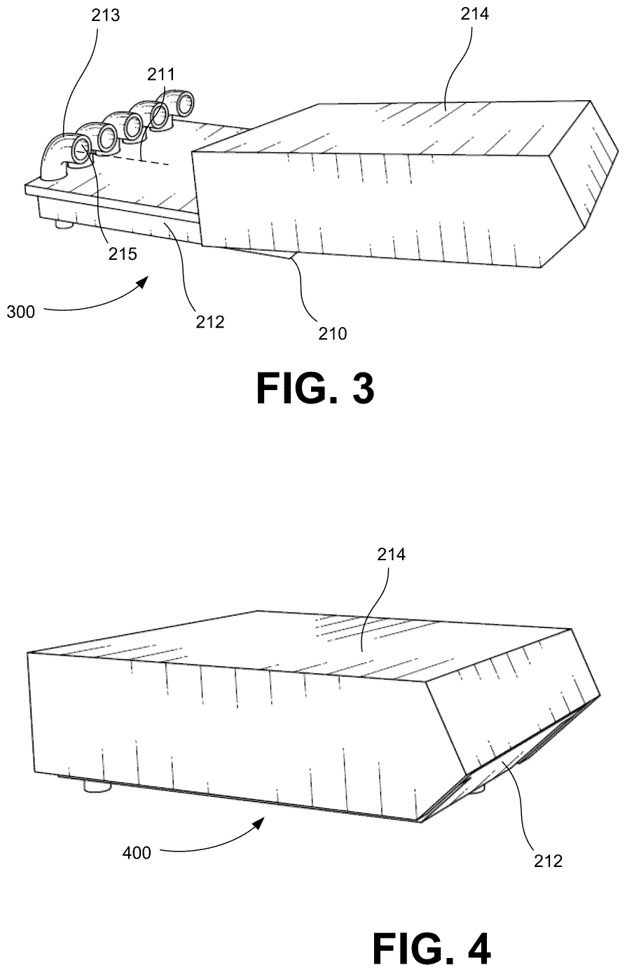

FIG. 3 is an exemplary perspective view 300 of the opened stool sample collector from the back and above, in accordance with various embodiments. In FIG. 3, platform 212 includes five pipes 213 extending out of platform 212 at a first end of platform 212. Each of five pipes 213 is shaped back towards platform 212 so that an axis 211 of opening 215 of each pipe of five pipes 213 is above platform 212, pointed back over platform 212, and parallel to platform 212.

Cover 214 is adapted to slide over platform 212 from a second end of platform 212 opposite the first end. At the second end of platform 212, cover 214 is positioned at an angle with respect to platform 212. This angle keeps cover 214 above the curved wall of the toilet bowl. This angle decreases as cover 214 is moved across platform 212 until the angle is zero and cover 214 is parallel to platform 212 in the closed position. Five pipes 213 are connected within platform 212 to a single pipe (not shown), for example, that extends out of platform 212 for attachment to a hose.

In various embodiments, stool sample collector 210 can be made of materials including, but not limited to, plastic, ceramic, porcelain, or stainless steel. In a preferred embodiment, for example, at least one or more pipes 213 is made of stainless steel. Stool sample collector 210 can be three-dimensionally (3D) printed or injection molded, for example.

FIG. 4 is an exemplary perspective view 400 of the closed stool sample collector from the back and above, in accordance with various embodiments. In FIG. 4, cover 214 has moved across platform 212 until the angle is zero, and cover 214 is parallel to platform 212 in the closed position. Note that at one end, both platform 212 and cover 214 are beveled to aid the flow of water around platform 212 and cover 214 during flushing.

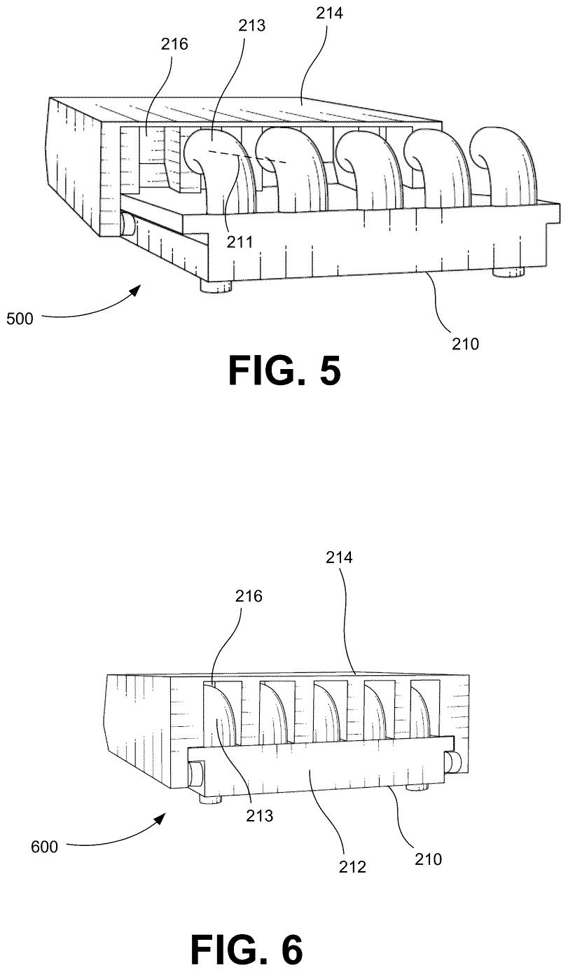

FIG. 5 is an exemplary diagram 500 showing a perspective view of the opened stool sample collector from the left and front, in accordance with various embodiments. FIG. 5 shows that cover 214 includes one or more surfaces 216 so that each surface of one or more surfaces 216 is perpendicular to axis 211 of one or more pipes 213. In other words, each surface of one or more surfaces 216 is designed to move stool toward each opening of one or more pipes 213 when stool sample collector 210 is in the process of being closed and to seal each opening of one or more pipes 213 when the stool sample collector is completely closed. This seal enables the stool to be suctioned from each pipe of one or more pipes 213 without also suctioning too much water from the toilet.

In various embodiments, each surface of one or more surfaces 216 is made of a soft or rubberized material to make a good seal with each pipe of one or more pipes 213.

FIG. 6 is an exemplary diagram 600 showing a perspective view of the closed stool sample collector from the left and back, in accordance with various embodiments. FIG. 6 shows that each surface of one or more surfaces 216 is placed in an indentation in cover 214 so that one or more pipes 213 are covered when stool sample collector 210 is completely closed. The walls of the indentations also help remove excess stool between one or more pipes 213 and from platform 212 as cover 214 is closing.

Returning to FIG. 2, one or more actuators 218 move cover 214 back and forth across platform 212. As shown in FIG. 2, in order to keep cover 214 above the surface of the curved toilet bowl, one or more actuators 218 also position cover 214 at an angle with respect to platform 212. As described above, this angle decreases as cover 214 is moved across platform 212 until the angle is zero and cover 214 is parallel to platform 212 in the closed position.

Although cover 214 is positioned at an angle with respect to platform 212, an edge of cover 214 is positioned to maintain contact with platform 212 so that it moves stool 201 towards one or more pipes 213 and, at the same time, cleans stool from platform 212 when it is moved backward across platform 212. In other words, cover 214 maintains a pivot point that is at the surface of platform 212 as cover 214 is moved and angled upward or downward.

To provide an area for stool collection, one or more processors 240 control one or more actuators 218 to move cover 214 to the second end of platform 212 providing an area of stool collection on platform 212 between cover 214 and one or more pipes 213. Stool 201 then lands on platform 212 between cover 214 and one or more pipes 213.

To move stool 201 toward and into one or more pipes 213, one or more processors 240 control one or more actuators 218 to move cover 214 back to the first end to move stool 201 collected on platform 212 toward and into openings of one or more pipes 213 until at least one of one or more surfaces 216 (shown in FIG. 5) is in contact with an opening of the one or more pipes 213.

Finally, to collect portions of stool 201, one or more processors 240 control one or more pumps 230 to provide suction to collection vessel 220 that, in turn, provides suction to one or more pipes 213 that move stool 201 from at least one pipe of one or more pipes 213 to collection vessel 220. One or more pumps 230 provide suction through second one or more hoses 231 to second port 222 of collection vessel 220. Collection vessel 220 then, in turn, provides suction to one or more pipes 213 through first port 221 and first one or more hoses 223.

In various embodiments, collection vessel 220 further includes overflow valve 224 to prevent liquid from flowing back towards one or more pumps 230. Second one or more hoses 231 can also include an air filter (not shown) to protect one or more pumps 230.

Note that FIG. 2 describes a mostly air suction system for suctioning stool and some water. In various alternative embodiments, the suction system can be designed as a liquid suctioning system for suctioning stool in water. One problem with a liquid suctioning system is that it collects a large volume of material, which is mostly water. In various embodiments, the material can be filtered, and excess water can be drained back into the toilet.

One or more processors 240 can be, but are not limited to, controllers, computers, microprocessors, computer systems as depicted by FIG. 1, or any devices capable of sending and receiving control signals or data and analyzing data. One or more processors 240 can include an Arduino or Raspberry Pi microcontroller, for example. One or more processors 240 send and receive control signals using one or more wires 242.

In various embodiments, one or more toilet seat sensors 250 detect that a person has sat on the toilet seat. One or more toilet seat sensors 250 are used to determine when to open stool sample collector 210. Specifically, one or more processors 240 control one or more actuators 218 to move cover 214 to the second end of platform 212 providing an area of stool collection on platform 212 between cover 214 and one or more pipes 213 when one or more toilet seat sensors 250 detect that a person has sat on the toilet seat.

In various embodiments, one or more toilet seat sensors 250 can include, but are not limited to, a mechanical switch, a Hall effect sensor, a strain gauge, an Infrared (IR) sensor, or a temperature sensor.

In various embodiments, stool sample collector 210 further includes one or more object detection sensors 260 for detecting stool 201 on platform 212. One or more object detection sensors 260 are used to determine when to close stool sample collector 210. Specifically, one or more processors 240 control one or more actuators 218 to move cover 214 to the first end to move stool 201 collected on platform 212 toward and into openings of one or more pipes 213 until at least one of one or more surfaces 216 (shown in FIG. 5) is in contact with an opening of one or more pipes 216 when one or more object detection sensors 260 detect stool 201 on platform 212.

In various embodiments, one or more object detection sensors 260 allow stool collection to be completed even while stool is still being produced and well before the toilet is flushed. As a result, a user may not even see the stool that has been collected unless they look in collection vessel 220.

In various embodiments, two or more of one or more object detection sensors 260 are used to determine an amount of stool 201 collected on platform 212. For example, if an array of five sensors of one or more object detection sensors 260 are used and four sensors detect stool 201, there is more confidence that the amount of stool 201 collected on platform 212 is sufficient for testing. The array can include, for example, a two-dimensional (2D) IR image detector.

In various embodiments, one or more object detection sensors 260 can include, but are not limited to, a mechanical switch, a Hall effect sensor, a strain gauge, an IR sensor, or a temperature sensor.

In various embodiments, one or more processors 240 control one or more pumps 230 to provide suction to collect stool 201 as soon as cover 214 has closed. Specifically, one or more processors 240 control one or more pumps 230 to provide suction to collection vessel 220 that, in turn, provides suction to one or more pipes 230 that moves stool 201 from at least one pipe of one or more pipes 213 to collection vessel 220 when cover 214 reaches the first end.

In various embodiments, one or more support structures 270 physically connect stool sample collector 210 to a rim of the toilet to fixate stool sample collector 210 in a location in the toilet. In FIG. 2, one or more support structures 270 are shown as a flat curved bracket secured by clamp 271 to the front rim of the toilet. In various embodiments, one or more support structures 270 can include, but are not limited to, flat curved brackets, bands, wires, rods, or tubes. One or more support structures 270 are, for example, made of stainless steel.

In various embodiments, stool sample collector 210 is easily removed temporarily for activities such as cleaning or plunging the toilet by simply opening clamp 271 and removing stool sample collector 210 along with one or more support structures 270.

In FIG. 2, one or more support structures 270 are shown as connected to the front rim of the toilet. In various embodiments, one or more support structures 270 can be connected to either side rim of the toilet or any part of the toilet.

In various embodiments, one or more support structures 270 are integrated into a toilet seat. In other words, the entire system for automatic stool sampling is part of a smart toilet seat. When the smart toilet seat is connected to the toilet, the system for automatic stool sampling is also connected.

In various embodiments, stool sample collector 214 is fixated underwater by one or more support structures 270. Keeping stool sample collector 214 underwater allows it to be cleaned through flushing action. It also prevents the release of any odors from any residual stool on stool sample collector 214. In various embodiments, stool sample collector 214 also includes weights to keep it underwater and in place. One or more support structures 270 provide support to prevent stool sample collector 214 from being moved by the water during flushing, for example.

In various embodiments, the system for automatic stool sampling also automatically cleans itself. For example, the freshwater supplied to the toilet can be used for cleaning.

To accomplish this, in various embodiments, the system for automatic stool sampling further includes three-way electrically controlled valve 280, hose 281, and tee fitting 282 for connecting to the water supply line of the toilet. Three-way electrically controlled valve 280 connects first port 221, first one or more hoses 223, and hose 281. At any one time, three-way electrically controlled valve 280 connects first port 221 to first one or more hoses 223 for suctioning stool or connects first port 221 to hose 281 for cleaning.

During stool collection, one or more processors 240 control three-way electrically controlled valve 280 to open the connection between first port 221 and first one or more hoses 223 when one or more processors control one or more pumps 230 to provide suction to collection vessel 220 that, in turn, provides suction to one or more pipes 213 that moves stool from at least one pipe of one or more pipes 213 to collection vessel 220.

During cleaning, one or more processors 240 control three-way electrically controlled valve 280 to open a connection between hose 281 and first one or more hoses 223 to send freshwater back out through one or more pipes 213 to clean first one or more hoses 223, one or more pipes 213, platform 212, and cover 214.

In various embodiments, the cover is moved while the freshwater is being used for cleaning. Specifically, one or more processors control 240 one or more actuators 218 to also move cover 214 toward the second end and back to the first end of platform 212 while one or more processors 240 control three-way electrically controlled valve 280 to be open between hose 281 and first one or more hoses 223.

In various embodiments, cleaning can also include purging first one or more hoses 223 of some water after cleaning by pumping air back through one or more hoses 223.

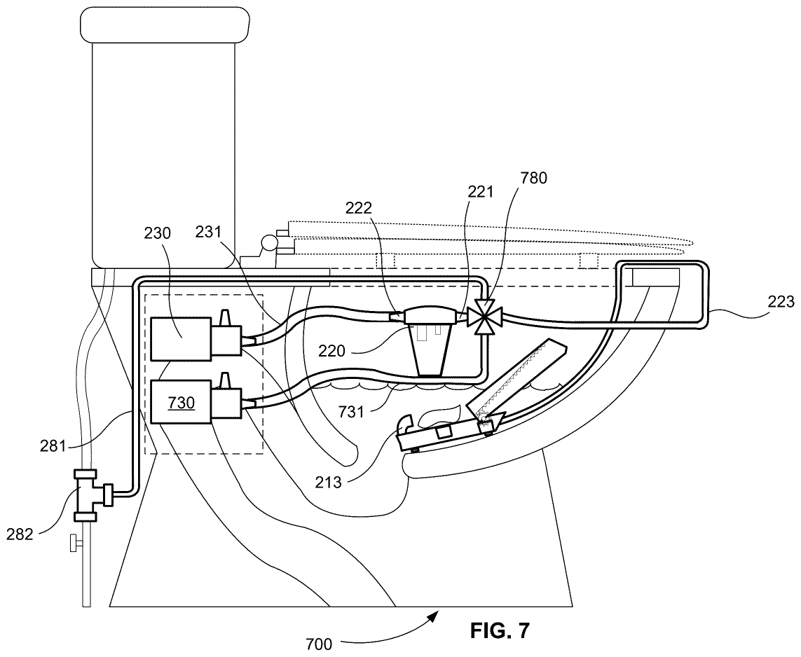

FIG. 7 is an exemplary diagram 700 showing that the system of FIG. 2 can include an additional pump for purging water from hoses, in accordance with various embodiments. Additional pump 730 is shown in FIG. 7. Pump 730 can be a pump of one or more pumps 230. FIG. 7 also shows four-way electrically controlled valve 780 that is connected to pump 730 through hose 731. Four-way electrically controlled valve 780 connects first port 221, first one or more hoses 223, hose 281, and hose 731. At any one time, four-way electrically controlled valve 780 connects first port 221 to first one or more hoses 223 for suctioning, connects hose 281 to first one or more hoses 223 for cleaning with fresh water, or connects hose 731 to first one or more hoses 223 for pumping air back through first one or more hoses 223.

To purge first one or more hoses 223 of water before stool collection or after cleaning, one or more processors 240 control four-way electrically controlled valve 780 to open a connection between hose 731 and first one or more hoses 223. One or more processors 240 then control pump 730 to pump air through hose 731 and on through first one or more hoses 223 to purge first one or more hoses 223 of at least some water. It may be desirable to purge first one or more hoses 223 of water to reduce the amount of water collected by collection vessel 220 during stool collection.

Returning to FIG. 2, in various embodiments, one or more processors 240 further include a wireless interface 241 for wirelessly connecting to smartphone 290 that allows smartphone 290 to control one or more processors 240 via a smartphone application running on smartphone 290. For example, collection vessel 220 includes a vessel barcode. The smartphone application then requires that the vessel barcode be scanned by smartphone 290 before a sample can be collected.

In various embodiments, smartphone 290 can also receive a notification when one or more toilet seat sensors 250 detect that a person has sat on the toilet seat. This allows a person to initiate a sample collection using the smartphone application

In various embodiments, one or more pumps 230 and one or more processors 240 are located in housing 205 attached to a side of the toilet. Housing 205 is attached to a side of the toilet using clamp 206, for example. Collection vessel 220 can be attached to or placed in housing 205.

In various embodiments, housing 205 includes a system barcode. The smartphone application then requires that the housing barcode be scanned by smartphone 290 before a sample can be collected.

In various embodiments, collection vessel 220 is connected to second one or more hoses 231 and three-way electrically controlled valve 280 using a quick connection and disconnection adapter (not shown) so that a user can quickly and easily change collection vessel 220.

In various embodiments, one or more actuators 218 can include, but are not limited to, a servo motor or stepper motor. As shown in FIG. 2, one or more actuators 218 include a continuous rotation servo motor, for example.

In various embodiments, one or more actuators 218 are waterproof.

In various embodiments, one or more actuators 218 can include actuators for moving stool sample collector 210 within the toilet bowl. For example, one or more actuators 218 can include an actuator for translating the stool sample collector in a direction parallel to the axis from the front of the toilet to the back of the toilet. In addition, one or more actuators 218 can include an actuator for translating the stool sample collector in a direction perpendicular to the axis from the front of the toilet to the back of the toilet. As a result, the position of stool sample collector 210 in the toilet can be adjusted manually or automatically.

In various embodiments, for automatic adjustment, one or more processors 240 can include a memory device and store information from the two or more of one or more object detection sensors 260 described above. If some sensors of an array of sensors do not sense stool over a large number of collections, then the position of stool sample collector 210 in the toilet is adjusted automatically to try to get more sensors to participate. The position of stool sample collector 210 in the toilet is adjusted by one or more processors 240 using actuators of one or more actuators 218 for moving stool sample collector 210 within the toilet bowl.

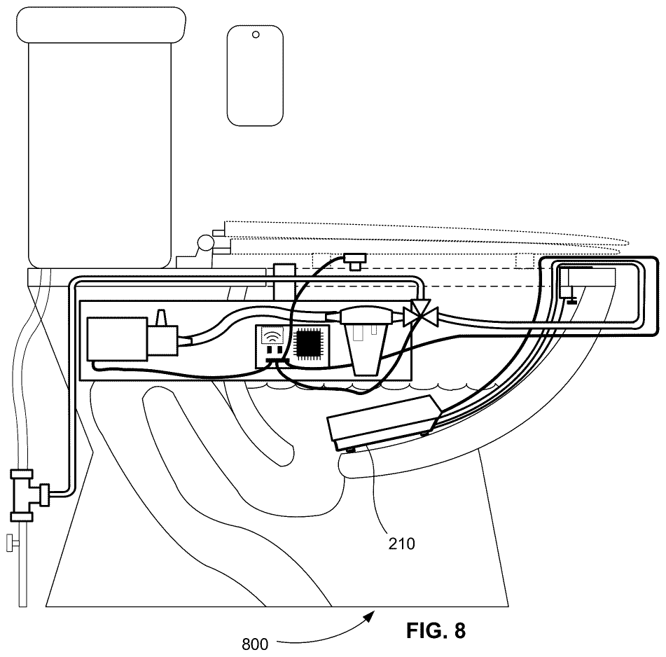

FIG. 8 is an exemplary diagram 800 showing the system of FIG. 2 after a sample has been collected, in accordance with various embodiments. In FIG. 8, stool sample collector 210 is shown in the closed position. In FIG. 8, stool sample collector 210 remains below the water line.

In various alternative embodiments, after a sample is collected, stool sample collector 210 can move up the toilet wall toward the rim of the toilet and above the water line. Returning to FIG. 2, stool sample collector 210 can move close to one or more support structures 270. Moving stool sample collector 210 moves it out of the way to avoid attracting toilet paper and other debris when not in use. stool sample collector 210 can be self-propelled or move along a track (not shown) between it and one or more support structures 270. The propelling motor can be in stool sample collector 210 or external to stool sample collector 210 when a track is used.

FIG. 9 is an exemplary perspective view 900 of the closed stool sample collector from the back and below, in accordance with various embodiments.

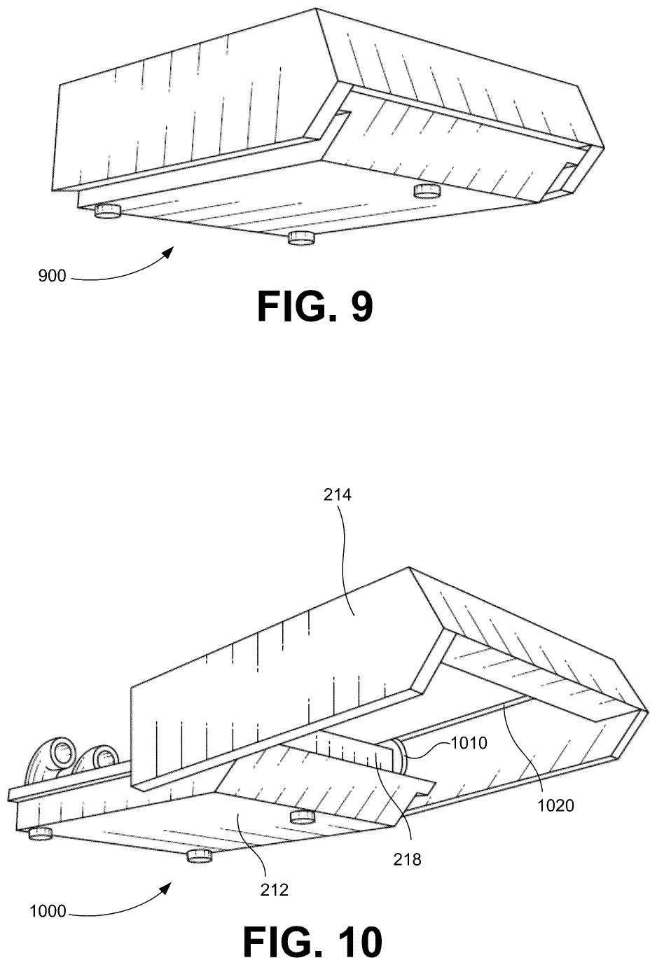

FIG. 10 is an exemplary perspective view 1000 of the opened stool sample collector from the back and below, in accordance with various embodiments. FIG. 10 shows an actuator of one or more actuators 218 located on platform 212 and used to move cover 214. The actuator of one or more actuators 218 uses cog 1010 moving along toothed track or groove 1020 in cover 214 to move and open cover 214. In various embodiments, toothed track 1020 is angled to angle cover 214.

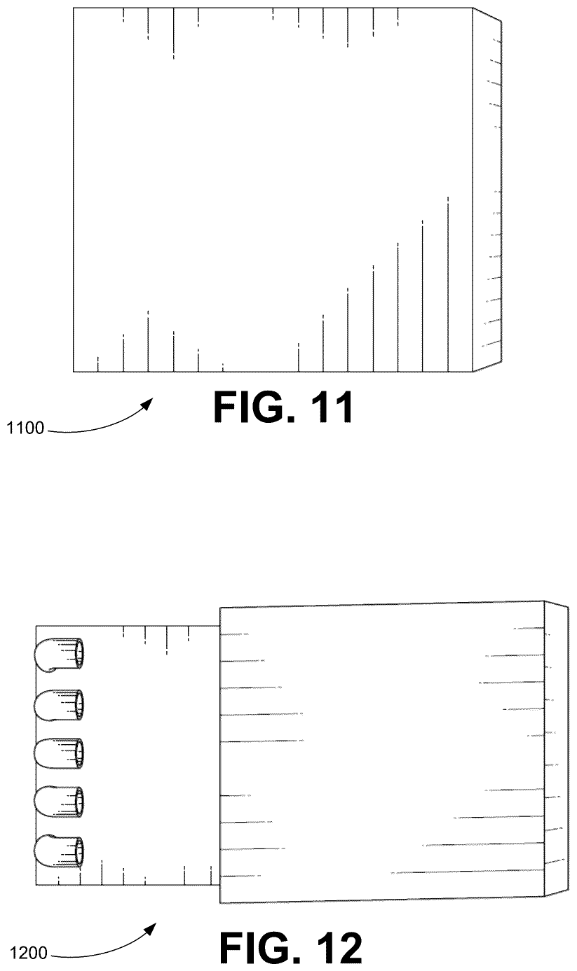

FIG. 11 is an exemplary perspective view 1100 of the closed stool sample collector from the top, in accordance with various embodiments.

FIG. 12 is an exemplary perspective view 1200 of the opened stool sample collector from the top, in accordance with various embodiments.

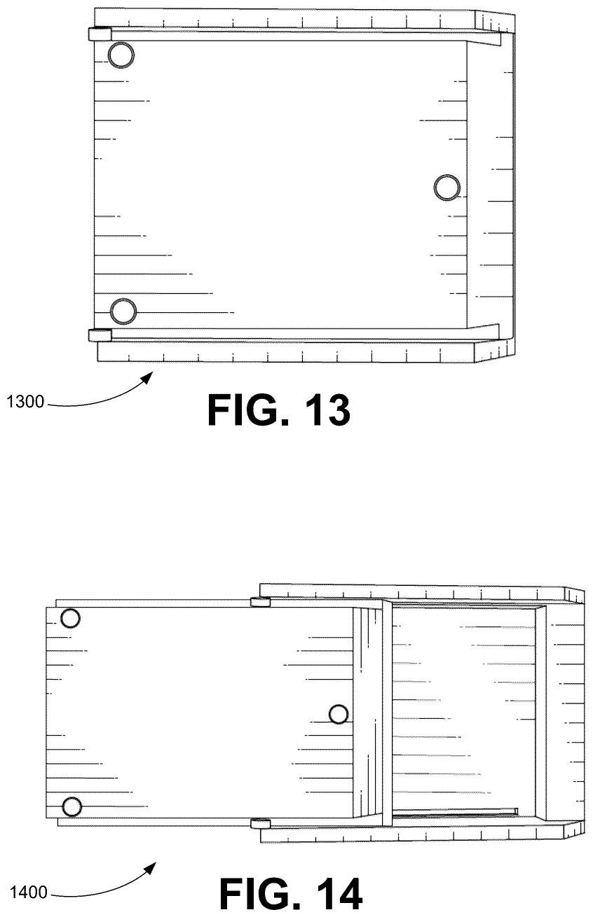

FIG. 13 is an exemplary perspective view 1300 of the closed stool sample collector from the bottom, in accordance with various embodiments.

FIG. 14 is an exemplary perspective view 1400 of the opened stool sample collector from the bottom, in accordance with various embodiments.

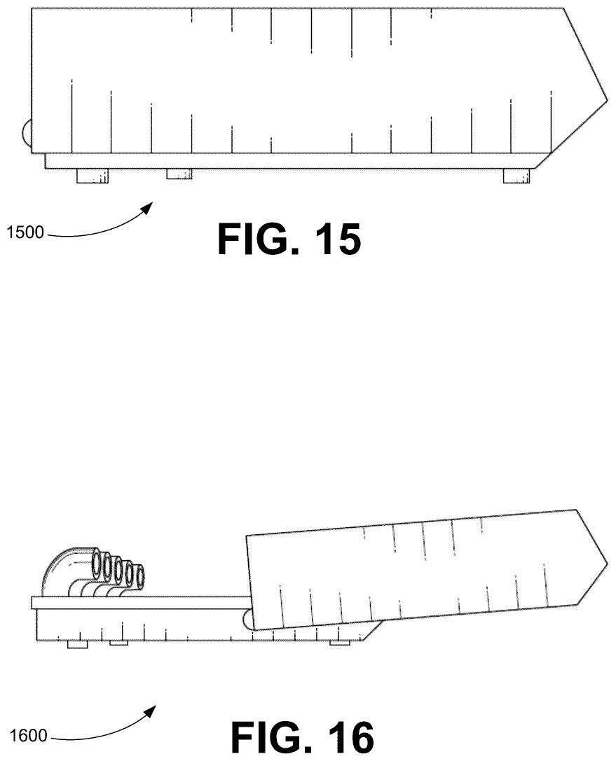

FIG. 15 is an exemplary perspective view 1500 of the closed stool sample collector from the right side, in accordance with various embodiments.

FIG. 16 is an exemplary perspective view 1600 of the opened stool sample collector from the right side, in accordance with various embodiments.

FIG. 17 is an exemplary perspective view 1700 of the closed stool sample collector from the left side, in accordance with various embodiments.

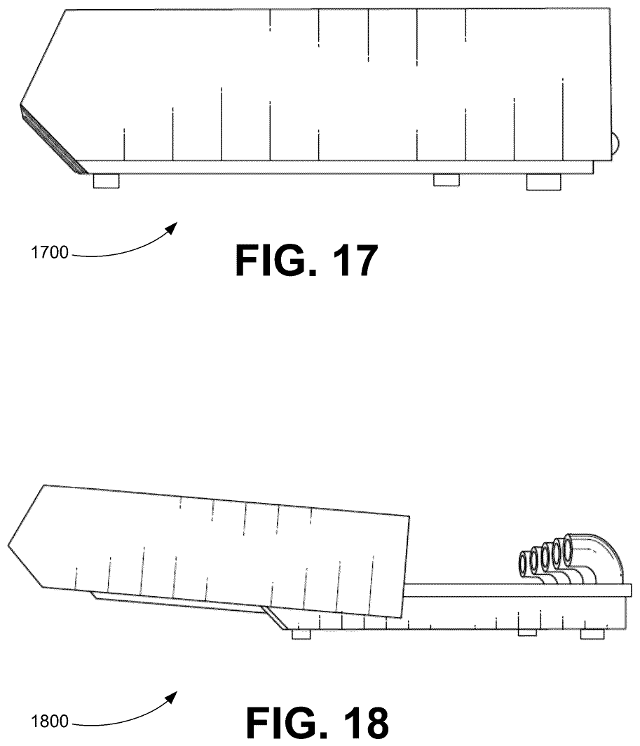

FIG. 18 is an exemplary perspective view 1800 of the opened stool sample collector from the left side, in accordance with various embodiments.

### Method for Automatic Stool Sampling

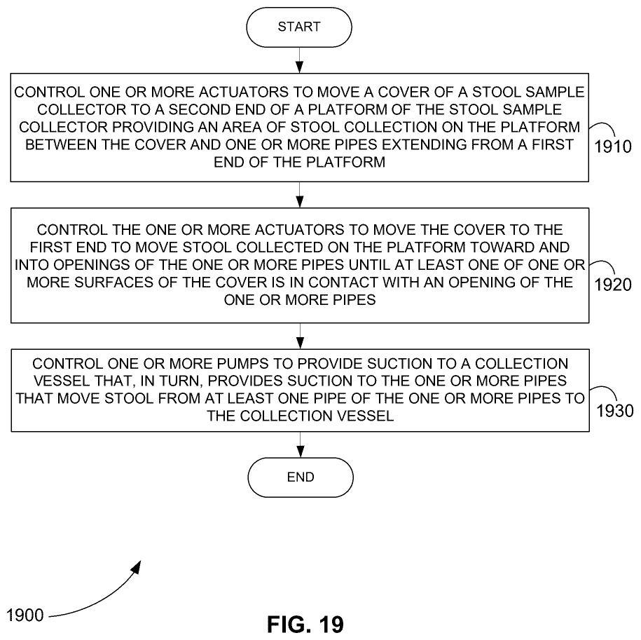

FIG. 19 is an exemplary flowchart showing a method 1900 for automatic stool sampling in a toilet, in accordance with various embodiments.

In step 1910 of method 1900, one or more actuators are controlled to move a cover of a stool sample collector to a second end of a platform of the stool sample collector providing an area of stool collection on the platform between the cover and one or more pipes extending from a first end of the platform.

In step 1920 the one or more actuators are controlled to move the cover to the first end to move stool collected on the platform toward and into openings of the one or more pipes until at least one of one or more surfaces of the cover is in contact with an opening of the one or more pipes; and

In step 1930, one or more pumps are controlled to provide suction to a collection vessel that, in turn, provides suction to the one or more pipes that move stool from at least one pipe of the one or more pipes to the collection vessel.

The stool sample collector is placed in a toilet. The one or more pipes extend out of the platform at the first end and are shaped back towards the platform so that an axis of an opening of each pipe of the one or more pipes is above the platform, pointed back over the platform, and parallel to the platform. The cover is adapted to slide over the platform from the second end opposite the first end and includes the one or more surfaces so that each surface of the one or more surfaces is perpendicular to axis of the one or more pipes. The one or more actuators move the cover back and forth across the platform. The collection vessel includes a first port connected to each of the one or more pipes through first one or more hoses. The one or more pumps are connected to a second port of the collection vessel through second one or more hoses.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Further, in describing various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments.

What is claimed is:

1. A system for automatic stool sampling in a toilet, comprising:
a stool sample collector placed in a toilet including a platform with one or more pipes extending out of the platform at a first end of the platform and shaped back towards the platform so that an axis of an opening of each pipe of the one or more pipes is above the platform, pointed back over the platform, and parallel to the platform, a cover that is adapted to slide over the platform from a second end of the platform opposite the first end and includes one or more surfaces so that each surface of the one or more surfaces is perpendicular to axis of the one or more pipes, and one or more actuators for moving the cover back and forth across the platform;
a collection vessel with a first port connected to each of the one or more pipes through first one or more hoses;
one or more pumps connected to a second port of the collection vessel through second one or more hoses; and
one or more processors that
controls the one or more actuators to move the cover to the second end of the platform providing an area of stool collection on the platform between the cover and the one or more pipes,
controls the one or more actuators to move the cover to the first end to move stool collected on the platform toward and into openings of the one or more pipes until at least one of the one or more surfaces is in contact with an opening of the one or more pipes, and
controls the one or more pumps to provide suction to the collection vessel that, in turn, provides suction to the one or more pipes that move stool from at least one pipe of the one or more pipes to the collection vessel.

2. The system of claim 1, further comprising one or more toilet seat sensors that detect that a person has sat on the toilet seat, wherein the one or more processors control the one or more actuators to move the cover to the second end of the platform providing an area of stool collection on the platform between the cover and the one or more pipes when the one or more toilet seat sensors detect that a person has sat on the toilet seat.

3. The system of claim 2, the one or more toilet seat sensors comprises a mechanical switch, a Hall effect sensor, a strain gauge, an Infrared (IR) sensor, or a temperature sensor.

4. The system of claim 1, wherein the stool sample collector further includes one or more object detection sensors for detecting stool on the platform and wherein the one or more processors control the one or more actuators to move the cover to the first end to move stool collected on the platform toward and into openings of the one or more pipes until at least one of the one or more surfaces is in contact with an opening of the one or more pipes when the one or more object detection sensors detect stool on the platform.

5. The system of claim 4, the one or more object detection sensors comprise a mechanical switch, a Hall effect sensor, a strain gauge, an Infrared (IR) sensor, or a temperature sensor.

6. The system of claim 1, wherein the one or more processors control the one or more pumps to provide suction to the collection vessel that, in turn, provides suction to the one or more pipes that moves stool from at least one pipe of the one or more pipes to the collection vessel when the cover reaches the first end.

7. The system of claim 1, further comprising one or more support structures that physically connect the stool sample collector to a rim of the toilet to fixate the stool sample collector in a location in the toilet.

8. The system of claim 7, wherein the stool sample collector is fixated underwater by the one or more support structures.

9. The system of claim 1, further comprising a three-way electrically controlled valve, a hose, and a tee fitting for connecting to the water supply line of the toilet, wherein the three-way electrically controlled valve connects the first port, the first one or more hoses, and the hose.

10. The system of claim 9, wherein the one or more processors control the three-way electrically controlled valve to open a connection between the first port and the first one or more hoses when the one or more processors control the one or more pumps to provide suction to the collection vessel that, in turn, provides suction to the one or more pipes that moves stool from at least one pipe of the one or more pipes to the collection vessel.

11. The system of claim 10, wherein the one or more processors control the three-way electrically controlled valve to open a connection between the hose and the first one or more hoses to send freshwater back out through the one or more pipes to clean the first one or more hoses, the one or more pipes, the platform, and the cover.

12. The system of claim 11, wherein the one or more processors control the one or more actuators to also move the cover toward the second end and back to the first end of the platform while the one or more processors control the three-way electrically controlled valve to be open between the hose and the first one or more hoses.

13. The system of claim 1, further a toilet seat that includes one or more support structures that physically connect the stool sample collector to the toilet seat to fixate the stool sample collector in a location in the toilet.

14. The system of claim 13, wherein the stool sample collector is fixated underwater.

15. The system of claim 1, wherein the one or more processors further include a wireless interface for wirelessly connecting to a smartphone that allows the smartphone to control the one or more processors via a smartphone application running on the smartphone.

16. The system of claim 15, wherein the collection vessel includes a vessel barcode and wherein the smartphone application requires that the vessel barcode be scanned by the smartphone before a sample can be collected.

17. The system of claim 16, wherein the one or more pumps and the one or more processors are located in a housing attached to a side of the toilet.

18. The system of claim 17, wherein the housing is attached to a toilet seat or to a rim of the toilet.

19. The system of claim 16, wherein the housing includes a system barcode and wherein the smartphone application requires scanning the system barcode.

20. A method for automatic stool sampling in a toilet, comprising:
controlling one or more actuators to move a cover of a stool sample collector to a second end of a platform of the stool sample collector providing an area of stool collection on the platform between the cover and one or more pipes extending from a first end of the platform;
controlling the one or more actuators to move the cover to the first end to move stool collected on the platform toward and into openings of the one or more pipes until at least one of one or more surfaces of the cover is in contact with an opening of the one or more pipes; and
controlling one or more pumps to provide suction to a collection vessel that, in turn, provides suction to the one or more pipes that move stool from at least one pipe of the one or more pipes to the collection vessel, wherein
the stool sample collector is placed in a toilet,
the one or more pipes extend out of the platform at the first end and are shaped back towards the platform so that an axis of an opening of each pipe of the one or more pipes is above the platform, pointed back over the platform, and parallel to the platform, wherein
the cover is adapted to slide over the platform from the second end opposite the first end and includes the one or more surfaces so that each surface of the one or more surfaces is perpendicular to axis of the one or more pipes,
the one or more actuators move the cover back and forth across the platform,
the collection vessel includes a first port connected to each of the one or more pipes through first one or more hoses, and
the one or more pumps are connected to a second port of the collection vessel through second one or more hoses.
